(19) United States
(12) Patent Application Publication
(10) Pub. No.: US 2017/0128622 A1
(43) Pub. Date: May 11, 2017

(54) MATERIALS AND METHODS FOR FILLING BONE VOIDS

(71) Applicant: 3-D MATRIX, LTD., Tokyo (JP)

(72) Inventors: Lisa Spirio, Lexington, MA (US); Eun Seok Gil, Lexington, MA (US)

(21) Appl. No.: 15/318,563

(22) PCT Filed: Jun. 19, 2015
(86) PCT No.: PCT/JP2015/003096

§ 371 Date: Dec. 13, 2016

Related U.S. Application Data
(60) Provisional application No. 62/015,151, filed on Jun. 20, 2014.

(51) Int. Cl. A61L 27/22; A61L 27/52; A61L 27/50

(52) U.S. Cl. CPC A61L 27/227; A61L 27/50; A61L 27/52; A61L 2430/02; A61L 2400/06; A61L 2400/12; A61F 2002/2839

(57) ABSTRACT

Materials and methods for bone void filling are provided. A peptide comprising between about 7 amino acids and about 32 amino acids in a solution may be introduced to a target site. The peptide may undergo self-organization under physiological conditions and/or in the presence of a cation.

### TECHNICAL FIELD

### Field of the Technology

One or more aspects relate generally to materials and methods that may be used in medical, research, and industrial applications. More particularly, one or more aspects relate to materials, such as membranes, hydrogels, compositions, and solutions, and methods that may be used to fill bone voids.

### SUMMARY OF INVENTION

### Solution to Problem

In accordance with one or more aspects, a method of filling a bone void in a subject is provided. The method may involve introducing a delivery device to a bone of a subject, positioning an end of the delivery device proximate a void in the bone where promotion of bone growth is desired, administering through the delivery device a solution comprising a self-assembling peptide comprising between about 7 and about 32 amino acids in an effective amount and in a concentration in a range of about 3 w/v percent to about 5 w/v percent peptide to form a hydrogel scaffold under physiological conditions to promote bone growth at the target site, and removing the delivery device from the subject.

In accordance with one or more further aspects, a method of filling a bone void in a subject is provided. The method may involve steps of introducing a delivery device to a bone of a subject, positioning an end of the delivery device proximate a void in the bone where promotion of bone growth is desired, administering through the delivery device a solution having a pH level of between about 3 and about 3.5, the solution comprising a self-assembling peptide comprising between about 7 and about 32 amino acids in an effective amount and in a concentration sufficient to form a hydrogel scaffold under physiological conditions to promote bone growth at the target site, and removing the delivery device from the subject.

In accordance with one or more aspects, a kit for filling a bone void in a subject is provided. The kit may include a solution comprising a self-assembling peptide comprising between about 7 amino acids and about 32 amino acids in an effective amount and in an effective concentration to form a hydrogel scaffold under physiological conditions to promote bone growth at a target site, and instructions for administering the solution to the target site in a bone of the subject.

Still other aspects and embodiments are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments.

### DESCRIPTION OF EMBODIMENTS

### Detailed Description

In accordance with one or more embodiments, materials and methods of the present disclosure may be used to fill bone voids. Beneficially, the disclosed materials and methods may be associated with greater mechanical strength, higher levels of biocompatibility, and more vital bone growth in comparison to conventional techniques.

In accordance with one or more specific embodiments, a peptide hydrogel may be used as a bone void filler (BVF) that resorbs and is replaced with bone during a healing process following administration at a target site. The peptide hydrogel may be placed into bony voids or gaps of the skeletal system. In certain embodiments, self-assembling peptides and self-assembled structures thereof may be used as cell culture supports for the repair and replacement of various tissues and as a scaffold to encapsulate living cells. The peptide hydrogel may promote tissue regeneration and the production of related extracellular matrix proteins. In at least some embodiments, the peptide hydrogel is non-immunogenic and represents an improvement over existing materials for this indication, including demineralized freeze-dried bone allograft (DFDBA) preparations.

The materials and methods may find particular application in filling various bone voids in a subject. As used herein, the term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the invention includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others.

In accordance with one or more embodiments, a target site may generally be any area or region in which promotion of bone growth is desired. In some embodiments, the target site may generally be associated with a surgical procedure. The target site may be located in any region of a bone of a subject. The target site may be a bone void. In some embodiments, the target site may be associated with an orthopedic condition or defect. A patient may be suffering from osteoporosis or a fracture. In some embodiments, calvarial bone defects may be addressed via bone healing. Cartilage or osteo-chondral effects may also be treated. Scar formation may be prevented. In some embodiments, the target site may be a position at which securing of an implant or prosthetic is desired so as to bridge any gap between an implant and surrounding tissue and to promote ingrowth into implants. In at least some embodiments, the development of bone tissue is facilitated with few or no gaps in a defect site.

In accordance with one or more embodiments, treatment may be accomplished in combination with one or more orthopedic medical devices and/or products. For example treatment with the materials of the present disclosure may be used in combination with orthobiologics, for example, bone graft substitutes, allograft distribution/processing, autogenous bone and soft tissue replacement products and viscoelastics, and bone growth stimulators. Treatment with the materials of the present disclosure may be used in combination with spinal implants or instrumentation, for example, internal fixation devices, discectomy and vertebroplasty/kyphoplasty products. Treatment with the materials of the present disclosure may be used in combination with orthopedic and dental implants; reconstructive surgery, for example, hip, knee, shoulder, elbow, wrist, ankle, and digit implants; fracture fixation, for example internal fixation and external fixation products; and arthroscopy/soft tissue repair, for example, scopes, camerals instruments, soft issue implants and repair kits.

In some embodiments, a periodontal disease is treated and/or dental tissue is regenerated. In certain embodiments, the dental tissue is periodontal ligament tissue. Exemplary periodontal diseases are periodontitis, gingivitis, periimplantitis and periimplant mucositis. In periodontitis, gums recede from the teeth and form pockets that become infected. Bacterial toxins and the immune system fighting the infection actually begin damaging the bone and connective tissue that hold teeth in place. Periimplantitis is a complication after surgical implantation of an alloplastic material into the jawbone and affects the tissues around an osseointegrated implant in function, resulting in loss of supporting bone. In certain embodiments, a therapeutically effective amount of a self-assembling peptide is administered to the periodontium. The periodontium consists of four tissues, gingival, periodontal ligament, cementum and alveolar bone. The gingiva is a pink-colored keratinized mucus membrane that covers parts of the teeth and part of the alveolar bone. The periodontal ligament is a group of connective tissue fibers that attach the tooth to alveolar bone. The cementum is a calcified structure that covers the lower parts of the teeth. The alveolar bone is a set of ridges from the jaw bones (maxillary and mandible) in which the teeth are embedded. The area where periodontal disease is initiated is the gingival sulcus, a pocket between the teeth and the gums. Dental bone content may be augmented at a target site in accordance with one or more embodiments. In some embodiments, the target site may be in an alveolar bone such as a posterior maxilla of the subject, commonly referred to as the upper jaw.

In accordance with one or more embodiments, a dental bone void may be filled as part of a procedure such a sinus lift, filling of a dental extraction socket, oral/maxillofacial augmentation or reconstruction, alveolar ridge augmentation, filling of a periodontal defects, and filling of a cystic defect.

The filling of a bone void may be partial or complete. In at least some embodiments, vital bone density may be increased at a target site. In some embodiments, the bone of a subject at a target site may be restored in part or in full. In various embodiments, a target site may be prepared such that an implant may be secured at the target site.

As discussed in greater detail below, the materials and methods may include the administration, application, or injection of a self-assembling peptide, or a solution comprising a self-assembling peptide, or a composition comprising a self-assembling peptide, to a predetermined or desired target area. In some non-limiting embodiments, the solution comprising a self-assembling peptide may be introduced into a dental bone void. Once the solution has been administered, the tissue may be closed such as surgically with stitches. A period of time, for example three to twelve months, may be allowed to elapse prior to implantation. This period of time may generally allow for a desired degree of bone growth and meshing in the bone void.

In accordance with one or more embodiments, peptide hydrogels may be used alone or in combination with one or more of autogenous bone, allografts, alloplasts, or xenografts. These combinations may generally increase the volume of graft material and may also improve overall performance. In at least some embodiments, methods may involve mixing the peptide solution with an autograft or an allograft prior to administration.

In accordance with one or more embodiments, a method of filling a bone void in a subject may involve introducing a delivery device to the subject. An end of the delivery device may be positioned proximate a target site in a bone of the subject where promotion of bone growth is desired. A solution comprising a self-assembling peptide comprising between about 7 and about 32 amino acids may be administered to the target site in an effective amount and in an effective concentration to form a hydrogel scaffold under physiological conditions to promote bone growth at the target site. The delivery device may then be removed from the subject.

In some embodiments, the concentration effective to promote bone growth comprises a concentration in a range of about 0.1 weight per volume (w/v) percent to about 5.0 w/v percent peptide. In some embodiments, the concentration may be in a range of about 1.0 w/v percent to about 5.0 w/v percent peptide. In at least some embodiments, the concentration may be in a range of about 3.0 w/v percent to about 5.0 w/v percent peptide.

In some embodiments, a pH level of a peptide solution may be effective to promote bone growth. In some embodiments, the pH level may be in a range of about 2 to about 3. In other embodiments, the pH level may be greater than 3, for example, between about 3 and about 3.5. In some non-limiting embodiments, the pH level may be about 3.4 or about 3.5.

The osmolality of the solution may determine the direction of water flow into or out of cells. In accordance with one or more embodiments, peptide solutions may be associated with osmolality levels such that solute concentration within and outside of cells are generally nearly about the same in order to avoid cell bursting, swelling, or other toxicity. In some embodiments, substantially isotonic solutions without net movement of water will generally not change cell volume, removing any possible instant toxicity in the surrounding cells, such as bone cells. In some embodiments, an osmolality level of a peptide solution may be between about 0 and about 300 mOsm/L. The osmolality of the peptide solution may be adjusted with one or more compounds including but not limited to dextrose, sucrose, lactose, manintol, glycerol, sodium, potassium, magnesium or calcium salts and any isotonicity adjuster. In some embodiments, the peptide solution may be adjusted up to about an isotonic osmolality level (i.e. around 300 mOsm/L). In other embodiments, the peptide solution may be adjusted up to about twice of an isotonic osmolality or higher with, for example, sodium chloride. The peptide solution may still be clear and injectable, without phase separation, even at the elevated osmolality levels discussed herein.

The administered volume may vary as discussed herein, for example, based on the dimensions of the target site and/or the desired degree of bone augmentation. In some non-limiting embodiments, the volume of the administered peptide solution is between about 1 mL and about 5 mL. In some specific embodiments, the administered peptide solution may be PuraMatrix® peptide hydrogel.

In some methods, an implant may be secured into augmented bone at the target site after a predetermined period of time. In accordance with one or more embodiments, a healing period ranging from a couple of months to a couple of years may be associated with a procedure to establish adequate bone regeneration at a target site. In some specific embodiments, healing of two months to one year may be required. In some embodiments, the predetermined period of time is between about three and about six months. In at least some embodiments, about six months of healing may be required. In some embodiments, additional doses of the peptide solution may be administered at the target site during the predetermined time period, randomly, upon visualization, or at regular intervals. In some embodiments, a supplemental volume of the peptide solution may be administered at the target site concurrently with implantation.

In other methods, an implant may be secured at the target site concurrently with administration of the peptide solution.

After administration, the target site may be surgically closed. A wound dressing may then be applied at the target site after administration of the peptide solution to facilitate healing and to help hold the peptide solution in place. The target site may be visualized after administration, such as at regular time intervals or after a predetermined period of time to assess bone augmentation.

In at least some embodiments, a self-assembled hydrogel scaffold at the target site may involve nanofibers having a diameter of about 10 nanometers to about 20 nanometers.

In accordance with one or more embodiments, the administered peptide solution is substantially non-biologically active. The disclosed methods may be associated with no IgG reaction. The resulting augmented bone may be characterized by a vital bone density of at least about 35% in some non-limiting embodiments.

The term “self-assembling peptide” may refer to a peptide that may exhibit a beta-sheet structure in aqueous solution in the presence of specific conditions to induce the beta-sheet structure. These specific conditions may include increasing the pH of a self-assembling peptide solution. The increase in pH may be an increase in pH to a physiological pH. The specific conditions may also include adding a cation, such as a monovalent cation, to a self-assembling peptide solution. The specific conditions may include conditions related to a mouth of a subject.

The self-assembling peptide may be an amphiphilic self-assembling peptide. By “amphiphilic” it is meant that the peptide comprises hydrophobic portions and hydrophilic portions. In some embodiments, an amphiphilic peptide may comprise, consist essentially of, or consist of alternating hydrophobic amino acids and hydrophilic amino acids. By alternating, it is meant to include a series of three or more amino acids that alternate between a hydrophobic amino acid and a hydrophilic amino acid, and it need not include each and every amino acid in the peptide sequence alternating between a hydrophobic and a hydrophilic amino acid. The self-assembling peptide, also referred to herein as “peptide” may be administered to the pre-determined or desired target area in the form of a self-assembling peptide solution, composition, hydrogel, membrane, scaffold or other form. The hydrogel may also be referred to as a membrane or scaffold throughout this disclosure. The predetermined or desired target area may be located in a bone of a subject, such as but not limited to alveolar bone.

The self-assembling peptide solution may be an aqueous self-assembling peptide solution. The self-assembling peptide may be administered, applied, or injected in a solution that is substantially cell-free, or free of cells. In certain embodiments, the self-assembling peptide may be administered, applied, or injected in a solution that is cell-free or free of cells.

The self-assembling peptide may also be administered, applied, or injected in a solution that is substantially drug-free or free of drugs. In certain embodiments, the self-assembling peptide may be administered, applied, or injected in a solution that is drug-free or free of drugs. In certain other embodiments, the self-assembling peptide may be administered, applied, or injected in a solution that is substantially cell-free and substantially drug-free. In still further certain other embodiments, the self-assembling peptide may be administered, applied, or injected in a solution that is cell-free and drug free.

The self-assembling peptide solution may comprise, consist of, or consist essentially of the self-assembling peptide. The self-assembling peptide may be in a modified or unmodified form. By modified, it is meant that the self-assembling peptide may have one or more domains that comprise one or more amino acids that, when provided in solution by itself, would not self-assemble. By unmodified, it is meant that the self-assembling peptide may not have any other domains other than those that provide for self-assembly of the peptide. That is, an unmodified peptide consists of alternating hydrophobic and hydrophilic amino acids that may self-assemble into a beta-sheet, and a macroscopic structure, such as a hydrogel.

Administration of a solution may comprise, consist of, or consist essentially of administration of a solution comprising, consisting of, or consisting essentially of a self-assembling peptide comprising, consisting of, or consisting essentially of between about 7 amino acids and about 32 amino acids. Other peptides that do not comprise, consist of, or consist essentially of between about 7 amino acids and about 32 amino acids may be contemplated by this disclosure.

By alternating, it is meant to include a series of three or more amino acids that alternate between a hydrophobic amino acid and a hydrophilic amino acid, and it need not include each and every amino acid in the peptide sequence alternating between a hydrophobic and a hydrophilic amino acid.

The materials and methods may comprise administering a self-assembling peptide to a predetermined or desired target. The peptide may be administered as a hydrogel or form a hydrogel upon administration. A hydrogel is a term that may refer to a colloidal gel that is dispersed in water. The hydrogel may also be referred to as a membrane or scaffold throughout this disclosure. The systems and methods may also comprise applying a self-assembling peptide to a predetermined or desired target as a solution such as an aqueous peptide solution.

The term “administering,” is intended to include, but is not limited to, applying, introducing, or injecting the self-assembling peptide, in one or more of various forms including, but not limited to, by itself, by way of solution, such as an aqueous solution, or by way of a composition, hydrogel, or scaffold, with or without additional components.

The method may comprise introducing a delivery device at or near a predetermined or desired target area of a subject. The method may comprise introducing a delivery device comprising at least one of a syringe, pipette, tube, catheter, syringe catheter, or other needle-based device to the predetermined or desired target area of a subject. The self-assembling peptide may be administered by way of a syringe, pipette, tube, catheter, syringe catheter, or other needle-based device to the predetermined or desired target area of a subject. The gauge of the syringe needle may be selected to provide an adequate flow of a composition, a solution, a hydrogel, or a liquid from the syringe to the target area. This may be based in some embodiments on at least one of the amount of self-assembling peptide in a composition, peptide solution, or a hydrogel being administered, the concentration of the peptide solution, in the composition, or the hydrogel, and the viscosity of the peptide solution, composition, or hydrogel. The delivery device may be a conventional device or designed to accomplish at least one of to reach a specific target area, achieve a specific dosing regime, deliver a specific target volume, amount, or concentration, and deliver accurately to a target area.

The disclosed methods of filling a bone void may comprise introducing a delivery device to a subject and positioning an end of the delivery device proximate the target site. Selective administration of the peptide may allow for enhanced and more targeted delivery of the peptide solution, composition, or hydrogel such that bone augmentation is successful and positioned in the desired location in an accurate manner. The selective administration may provide enhanced, targeted delivery that markedly improves the positioning and effectiveness of the treatment over conventional delivery devices. Delivery devices that may be used in the systems, methods, and kits of the disclosure may include a syringe, pipette, tube, catheter, syringe catheter, other needle-based device, tube or catheter.

Use of the delivery device may include use of accompanying devices, such as a guidewire used to guide the device into position, or an endoscope that may allow proper placement and visualization of the target area, and/or the path to the target area. The endoscope may be a tube that may comprise at least one of a light and a camera or other visualization device to allow images of the subject's body to be viewed.

The use of the delivery device, such as a syringe, pipette, tube, catheter, syringe catheter, other needle-based device, catheter, or endoscope may require determining the diameter or size of the opening in which there is a target area, such that at least a portion of the syringe, pipette, tube, syringe catheter, other needle-type device, catheter, or endoscope may enter the opening to administer the peptide, peptide solution, composition, or hydrogel to the target area.

In certain embodiments, the hydrogel may be formed in vitro and administered to the desired location in vivo. In certain examples, this location may be the area in which it is desired to promote bone growth. In other examples, this location may be upstream, downstream of the area, or substantially near the area. It may be desired to allow a migration of the hydrogel to the area in which it is desired to promote bone growth. Alternatively, another procedure may position the hydrogel in the area in which it is desired. The desired location or target area may be at least a portion of an area associated with a surgical procedure.

In certain aspects of the disclosure, the hydrogel may be formed in vivo. A solution comprising the self-assembling peptide, such as an aqueous solution, may be inserted to an in vivo location or area of a subject to prevent or reduce an obstruction or prevent or reduce a stenosis at that location. In certain examples, the hydrogel may be formed in vivo at one location, and allowed to migrate to the area in which it is desired to promote bone growth. Alternatively, another procedure may place the hydrogel in the area in which it is desired to promote bone growth. The peptides of the present disclosure may be in the form of a powder, a solution, a gel, or the like. Since the self-assembling peptide gels in response to changes in solution pH and salt concentration, it can be distributed as a liquid that gels upon contact with a subject during application or administration.

In certain environments, the peptide solution may be a weak hydrogel and, as a result, it may be administered by way of a delivery device as described herein.

In accordance with one or more embodiments, self-assembling peptides may promote bone growth. In certain embodiments, this may be because the hydrogel, once in place, provides a scaffold to allow for an infiltration of cells that promote bone growth of the target area.

In accordance with one or more embodiments, a macroscopic scaffold is provided. The macroscopic scaffold may comprise, consist essentially of, or consist of a plurality of self-assembling peptides, each of which comprises, consists essentially of, or consists of between about 7 amino acids and about 32 amino acids in an effective amount that is capable of being positioned within a dental bone void to promote bone growth therein.

In accordance with some embodiments, the self-assembling peptides may be amphiphilic, alternating between hydrophobic amino acids and hydrophilic amino acids. In accordance with one or more embodiments, a subject may be evaluated to determine a need for dental bone augmentation. Once the evaluation has been completed, a peptide solution to administer to the subject may be prepared.

In some embodiments, a biologically active agent may be used with the materials and methods of the present disclosure. A biologically active agent may comprise a compound, including a peptide, DNA sequence, chemical compound, or inorganic or organic compound that may impart some activity, regulation, modulation, or adjustment of a condition or other activity in a subject or in a laboratory setting. The biologically active agent may interact with another component to provide such activity. The biologically active agent may be referred to as a drug in accordance with some embodiments herein. In certain embodiments, one or more biologically active agents may be gradually released to the outside of the peptide system. For example, the one or more biologically active agents may be gradually released from the hydrogel. Both in vitro and in vivotesting has demonstrated this gradual release of a biologically active agent. The biologically active agent may be added to the peptide solution prior to administering to a subject, or may be administered separately from the solution to the subject.

This disclosure relates to aqueous solutions, hydrogels, scaffolds, and membranes comprising self-assembling peptides, sometimes referred to as self-assembling oligopeptides. The peptides may be comprised of a peptide having about 6 to about 200 amino acid residues. The self-assembling peptides may exhibit a beta-sheet structure in aqueous solution in the presence of physiological pH and/or a cation, such as a monovalent cation, or other conditions applicable to the mouth of a subject. The peptides may be amphiphilic and alternate between a hydrophobic amino acid and a hydrophilic amino acid. In certain embodiments, the peptide may comprise a first portion that may be amphiphilic, alternating between a hydrophobic amino acid and a hydrophilic amino acid, and another portion or region that is not amphiphilic.

The peptides may be generally stable in aqueous solutions and self-assemble into large, macroscopic structures, scaffolds, or matrices when exposed to physiological conditions, neutral pH, or physiological levels of salt. Once the hydrogel is formed it may not decompose, or may decompose or biodegrade after a period of time. The rate of decomposition may be based at least in part on at least one of the amino acid sequence and conditions of its surroundings.

By “macroscopic” it is meant as having dimensions large enough to be visible under magnification of 10-fold or less. In preferred embodiments, a macroscopic structure is visible to the naked eye. A macroscopic structure may be transparent and may be twodimensional, or three-dimensional. Typically each dimension is at least 10 μm, in size. In certain embodiments, at least two dimensions are at least 100 μm, or at least 1000 μm in size. Frequently at least two dimensions are at least 1-10 mm in size, 10-100 mm in size, or more.

In certain embodiments, the size of the filaments may be about 10 nanometers (nm) to about 20 nm. The interfilament distance may be about 50 nm to about 80 nm.

“Physiological conditions” may occur in nature for a particular organism, cell system, or subject which may be in contrast to artificial laboratory conditions. The conditions may comprise one or more properties such as one or more particular properties or one or more ranges of properties. For example, the physiological conditions may include a temperature or range of temperatures, a pH or range of pH's, a pressure or range of pressures, and one or more concentrations of particular compounds, salts, and other components. For example, in some examples, the physiological conditions may include a temperature in a range of about 20 to about 40 degrees Celsius. In some examples, the atmospheric pressure may be about 1 atm. The pH may be in the range of a neutral pH. For example, the pH may be in a range of about 6 to about 8. The physiological conditions may include cations such as monovalent metal cations that may induce membrane or hydrogel formation. These may include sodium chloride (NaCl). The physiological conditions may also include a glucose concentration, sucrose concentration, or other sugar concentration, of between about 1 mM and about 20 mM. The physiological conditions may vary with bone location.

In some embodiments, the self-assembling peptides may be peptides of between about 6 amino acids and about 200 amino acids. In certain embodiments, the self-assembling peptides may be peptides of at least about 7 amino acids. In certain embodiments, the self-assembling peptides may be peptides of between about 7 amino acids and about 32 amino acids. In certain further embodiments, the self-assembling peptides may be peptides of between about 7 amino acids and about 17 amino acids. In certain other examples, the self-assembling peptides may be peptides of at least 8 amino acids, at least about 12 amino acids, or at least about 16 amino acids.

The peptides may also be complementary and structurally compatible. Complementary refers to the ability of the peptides to interact through ionized pairs and/or hydrogen bonds which form between their hydrophilic side-chains, and structurally compatible refers to the ability of complementary peptides to maintain a constant distance between their peptide backbones. Peptides having these properties participate in intermolecular interactions which result in the formation and stabilization of beta-sheets at the secondary structure level and interwoven filaments at the tertiary structure level.

Both homogeneous and heterogeneous mixtures of peptides characterized by the above-mentioned properties may form stable macroscopic membranes, filaments, and hydrogels. Peptides which are self-complementary and self-compatible may form membranes, filaments, and hydrogels in a homogeneous mixture. Heterogeneous peptides, including those which cannot form membranes, filaments, and hydrogels in homogeneous solutions, which are complementary and/or structurally compatible with each other may also self-assemble into macroscopic membranes, filaments, and hydrogels.

The membranes, filaments, and hydrogels may be non-cytotoxic. The hydrogels of the present disclosure may be digested and metabolized in a subject. The hydrogels may be biodegraded in 30 days or less. They have a simple composition, are permeable, and are easy and relatively inexpensive to produce in large quantities. The membranes and filaments, hydrogels or scaffolds may also be produced and stored in a sterile condition. The optimal lengths for membrane formation may vary with at least one of the amino acid composition, solution conditions, and conditions at the target site.

In certain embodiments, a method of filling a bone void in a subject is provided. The method may comprise introducing a delivery device proximate a target site of a subject where promotion of bone growth is desired. The method may further comprise administering through the delivery device a solution comprising a self-assembling peptide comprising between about 7 amino acids and about 32 amino acids in an effective amount and in an effective concentration to form a hydrogel scaffold under physiological conditions to promote bone growth at the target site. The method may further comprise removing the delivery device from the subject.

The method may further comprise visualizing a region or target area comprising at least a portion of the bone. Visualizing the region or target area may comprise visualizing the region or target area during at least one of identifying the target area, introducing the delivery device, positioning the end of the delivery device in the target area, administering the solution, removing the delivery device, and monitoring the target site thereafter. Visualizing the region or target area may provide for selective administration of the solution. Visualizing may occur at any time before, during, and after the administration of the solution. Visualization may occur, for example, at a time period of at least one of about one week subsequent to administration, about four weeks subsequent to administration and about eight weeks subsequent to administration.

The solution to be administered may consist essentially of, or consist of, a self-assembling peptide comprising at least about 7 amino acids. The solution to be administered may consist essentially of, or consist of, a self-assembling peptide comprising between about 7 amino acids and about 32 amino acids. The peptide may be amphiphilic and at least a portion of the peptide may alternate between a hydrophobic amino acid and a hydrophilic amino acid.

Methods of facilitating embodiments of the present disclosure may comprise providing instructions for administering through a delivery device a solution comprising a self-assembling peptide comprising between about 7 amino acids and about 32 amino acids in an effective amount and in an effective concentration to form a hydrogel under physiological conditions to promote bone growth. The peptide may be amphiphilic and at least a portion of the peptide may alternate between a hydrophobic amino acid and a hydrophilic amino acid.

The methods of facilitating may comprise providing the solution comprising a self-assembling peptide comprising between about 7 amino acids and about 32 amino acids in an effective amount and in an effective concentration to form a hydrogel under physiological conditions to promote bone growth. The peptide may be amphiphilic and at least a portion of the peptide may alternate between a hydrophobic amino acid and a hydrophilic amino acid.

The methods of facilitating may comprise providing instructions to visualize a region or target area comprising at least a portion of the subject bone. The method may comprise providing instructions to visualize the target area or region during at least one of identifying the target area, introducing a delivery device, positioning an end of the delivery device in the target area, administering the solution, removing the delivery device, and monitoring thereafter. The method may comprise providing instructions to visualize the target area in a time period about one week, about four weeks, or about eight weeks subsequent to the administration. Instructions may be provided to monitor the area at the target area or surrounding the target area. Instructions may be provided to use the methods of the present disclosure during or after a surgical procedure.

The amino acids of the self-assembling or amphiphilic peptides may be selected from d-amino acids, 1-amino acids, or combinations thereof. The hydrophobic amino acids may include Ala, Val, Ile, Met, Phe, Tyr, Trp, Ser, Thr and Gly. The hydrophilic amino acids may be basic amino acids, for example, Lys, Arg, His, Orn; acidic amino acids, for example, Glu, Asp; or amino acids which form hydrogen bonds, for example, Asn, Gln. Acidic and basic amino acids may be clustered on a peptide. The carboxyl and amino groups of the terminal residues may be protected or not protected. Membranes or hydrogels may be formed in a homogeneous mixture of self-complementary and self-compatible peptides or in a heterogeneous mixture of peptides which are complementary and structurally compatible to each other. Peptides fitting the above criteria may self-assemble into macroscopic membranes under suitable conditions, described herein.

The self-assembling peptides may be composed of about 6 to about 200 amino acid residues. In certain embodiments, about 7 to about 32 residues may be used in the self-assembling peptides, while in other embodiments self-assembling peptides may have about 7 to about 17 residues. The peptides may have a length of about 5 nm.

The peptides of the present disclosure may include peptides having the repeating sequence of arginine, alanine, aspartic acid and alanine (Arg-Ala-Asp-Ala (RADA)), and such peptide sequences may be represented by (RADA)ₚ, wherein p=2-50 such as (RADA)₄ or RADA16 (i.e. RADARADARADARADA).

Other peptide sequences may be represented by self-assembling peptides having the repeating sequence of isoleucine, glutamic acid, isoleucine and lysine (Ile-Glu-Ile-Lys (IEIK), and such peptide sequences are represented by (IEIK)ₚ, wherein p=2-50, such as IEIK13. Other peptide sequences may be represented by self-assembling peptides having the repeating sequence of isoleucine, glutamic acid, isoleucine and lysine (Ile-Glu-Ile-Lys (IEIK), and such peptide sequences are represented by (IEIK)ₚI, wherein p=2-50.

Other peptide sequences may be represented by self-assembling peptides having the repeating sequence of lysine, leucine, aspartic acid, and leucine (Lys-Leu-Asp-Leu (KLDL)), and such peptide sequences are represented by (KLDL)ₚ, wherein p=2-50. Other peptide sequences may be represented by self-assembling peptides having the repeating sequence of lysine, leucine, and aspartic acid (Lys-Leu-Asp (KLD)), and such peptide sequences are represented by (KLD)ₚ, wherein p=2-50. As specific examples of self-assembling peptides according to the invention there may be a self-assembling peptide RADA16 having the sequence Arg-Ala-Asp-Ala-Arg-Ala-Asp-Ala-Arg-Ala-Asp-Ala-Arg-Ala-Asp-Ala (RADA)₄, a self-assembling peptide IEIK13 having the sequence Ile-Glu-Ile-Lys-Ile-Glu-Ile-Lys-Ile-Glu-Ile-Lys-Ile (IEIK)₃I, a self-assembling peptide IEIK17 having the sequence Ile-Glu-Ile-Lys-Ile-Glu-Ile-Lys-Ile-Glu-Ile-Lys-Ile-Glu-Ile-Lys-Ile (IEIK)₄I or a self-assembling peptide KLDL12 having the sequence Lys-Leu-Asp-Leu-Lys-Leu-Asp-Leu-Lys-Leu-Asp-Leu (KLDL)₃.

Each of the peptide sequences disclosed herein may provide for peptides comprising, consisting essentially of, and consisting of the amino acid sequences recited.

The present disclosure provides materials, methods, and kits for solutions, hydrogels, and scaffolds comprising, consisting essentially of, or consisting of the peptides recited herein.

A 1 weight per volume (w/v) percent aqueous (water) solution and a 2.5 w/v percent of (RADA)₄ is commercially available as the product PuraMatrix® peptide hydrogel offered by 3-D Matrix Co., Ltd.

Certain peptides may contain sequences which are similar to the cell attachment ligand RGD (Arginine-Glycine-Aspartic acid). The RAD-based peptides may be of particular interest because the similarity of this sequence to RGD. The RAD sequence is a high affinity ligand present in the extracellular matrix protein tenascin and is recognized by integrin receptors.

The self-assembly of the peptides may be attributable to hydrogen bonding and hydrophobic bonding between the peptide molecules by the amino acids composing the peptides.

The self-assembling peptides of the present disclosure may have a nanofiber diameter in a range of about 10 nm to about 20 nm and an average pore size is in a range of about 5 nm to about 200 nm. In certain embodiments, the nanofiber diameter, the pore size, and the nanofiber density may be controlled by at least one of the concentration of peptide solution used and the amount of peptide solution used, such as the volume of peptide solution. As such, at least one of a specific concentration of peptide in solution and a specific amount of peptide solution to provide at least one of a desired nanofiber diameter, pore size, and density to adequately provide for bone growth may be selected.

As used herein, an amount of a peptide, peptide solution or hydrogel effective to promote bone growth, an “effective amount” or a “therapeutically effective amount,” refers to an amount of the peptide, peptide solution or hydrogel, which is effective, upon single or multiple administration (application or injection) to a subject, in augmenting, treating, or in curing, alleviating, relieving or improving a subject with a bone void or other disorder beyond that expected in the absence of such treatment. This may include a particular concentration or range of concentrations of peptide in the peptide solution or hydrogel and additionally, or in the alternative, a particular volume or range of volumes of the peptide solution or hydrogel. The method of facilitating may comprise providing instructions to prepare at least one of the effective amount and the effective concentration.

The dosage, for example, volume or concentration, administered (for example, applied or injected) may vary depending upon the form of the peptide (for example, in a peptide solution, hydrogel, or in a dried form, such as a lyophilized form) and the route of administration utilized. The exact formulation, route of administration, volume, and concentration can be chosen in view of the subject's condition and in view of the particular target area or location that the peptide solution, hydrogel, or other form of peptide will be administered. Lower or higher doses than those recited herein may be used or required. Specific dosage and treatment regimens for any particular subject may depend upon a variety of factors, which may include the specific peptide or peptides employed, the dimension of the area that is being treated, the desired thickness of the resulting hydrogel that may be positioned in the desired target area, and the length of time of treatment. Other factors that may affect the specific dosage and treatment regimens include age, body weight, general health status, sex, time of administration, rate of degradation, the severity and course of the disease, condition or symptoms, and the judgment of the treating physician. In certain embodiments, the peptide solution may be administered in a single dose. In other embodiments, the peptide solution may be administered in more than one dose, or multiple doses. The peptide solution may be administered in at least two doses.

An effective amount and an effective concentration of the peptide solution may be selected to at least partially augment bone growth in a bone void. In some embodiments, at least one of the effective amount and the effective concentration may be based in part on a dimension or diameter of the target area.

The effective amount may be, as described herein, an amount that may provide for an at least partial augmentation of bone. Various properties in the bone region of the patient may contribute to the selection or determination of the effective amount including at least one of the dimension or diameter of the target area, the flow rate of one or more fluids at or near the target area, the pH at or near the target area, and the concentration of various salts at or near the target area. Additional properties that may determine the effective amount include various properties listed above, at various locations along a pathway in which the peptide solution is delivered.

The effective amount may include volumes of from about 0.1 milliliters (mL) to about 100 mL of a peptide solution. The effective amount may include volumes of from about 0.1 mL to about 10 mL of a peptide solution. The effective amount may include volumes of from about 1 mL to about 5 mL of a peptide solution. In certain embodiments, the effective amount may be about 0.5 mL. In other embodiments, the effective amount may be about 1.0 mL. In yet other embodiments, the effective amount may be about 1.5 mL. In still yet other embodiments, the effective amount may be about 2.0 mL. In some other embodiments, the effective amount may be about 3.0 mL. In certain embodiments, the effective amount may be approximately 0.1 mL to about 5 mL per 1 cm² of target area. In certain embodiments, the effective amount may be approximately 1 mL per 1 cm² of target area. This effective amount may be related to a concentration, such as a 2.5 weight per volume percent of a peptide solution of the present disclosure.

In some embodiments, a more effective bone augmentation may be achieved with a greater volume of peptide solution administered or a higher concentration of peptide in solution to be administered. This may allow a longer lasting or thicker hydrogel to form within the target area, allowing a more secure position of the hydrogel in the target area. It is possible that if a high enough volume is not selected, the hydrogel may not be effective at the target area for the desired period of time.

The effective concentration may be, as described herein, an amount that may provide for a desired level of bone augmentation. Various properties of the target site may contribute to the selection or determination of the effective concentration including at least one of a dimension or diameter of the target area.

The effective concentration may include peptide concentrations in the solution in a range of about 0.1 w/v percent to about 3.0 w/v percent. In certain embodiments, the effective concentration may be about 1 w/v percent. In other embodiments, the effective concentration may be about 2.5 w/v percent. In still other embodiments, the effective concentration may be between about 3.0 w/v percent and about 5.0 w/v percent.

In at least some embodiments, a stock solution of PuraMatrix® (1% w/v) may have a pH level of about 2.0 to about 3.0. In some embodiments, a peptide solution may have a pH level of at least 3, such as between about 3.0 and about 3.5, for example, about 3.4 or about 3.5.

In certain embodiments, a peptide solution having a higher concentration of peptide may provide for a more effective hydrogel that has the ability to stay in place and provide effective bone growth. For purposes of delivering the peptide solution, higher concentrations of peptide solutions may become too viscous to allow for effective and selective administration of the solution. It is possible that if a high enough concentration is not selected, the hydrogel may not be effective at promoting bone growth at the target area for the desired period of time. The effective concentration may be selected to provide for a solution that may be administered by injection or other means using a particular diameter needle or other delivery device.

Methods of the disclosure contemplate single as well as multiple administrations of a therapeutically effective amount of the peptides, compositions, peptide solutions, membranes, filaments, and hydrogels as described herein. Peptides as described herein may be administered at regular intervals, depending on the nature, severity and extent of the subject's condition. In some embodiments, a peptide, composition, peptide solution, membrane, filament, or hydrogel may be administered in a single administration. In some embodiments, a peptide, composition, peptide solution, or hydrogel described herein is administered in multiple administrations. In some embodiments, a therapeutically effective amount of a peptide, composition, peptide solution, membrane, filament, or hydrogel may be administered periodically at regular intervals. The regular intervals selected may be based on any one or more of the initial peptide concentration of the solution administered, the amount administered, and the degradation rate of the hydrogel formed. For example, after an initial administration, a follow-on administration may occur after, for example, one week, two weeks, four weeks, six weeks, or eight weeks. The follow-on administration may comprise administration of a solution having the same concentration of peptide and volume as the initial administration, or may comprise administration of a solution of lesser or great concentration of peptide and volume. The selection of the appropriate follow-on administration of peptide solution may be based on imaging the target area and the area surrounding the target area and ascertaining the needs based on the condition of the subject. The predetermined intervals may be the same for each follow-on administration, or they may be different. This may be dependent on whether the hydrogel formed from the previous administration is partially or totally disrupted or degraded. The follow-on administration may comprise administration of a solution having the same concentration of peptide and volume as the initial administration, or may comprise administration of a solution of lesser or great concentration of peptide and volume. The selection of the appropriate follow-on administration of peptide solution may be based on imaging the target area and the area surrounding the target area and ascertaining the needs based on the condition of the subject.

The self-assembling peptides of the present disclosure, such as RADA16, may be peptide sequences that lack a distinct physiologically or biologically active motif or sequence, and therefore may not impair intrinsic cell function. Physiologically active motifs may control numerous intracellular phenomena such as transcription, and the presence of physiologically active motifs may lead to phosphorylation of intracytoplasmic or cell surface proteins by enzymes that recognize the motifs. When a physiologically active motif is present, transcription of proteins with various functions may be activated or suppressed. The self-assembling peptides of the present disclosure may lack such physiologically active motifs and therefore do not carry this risk. A sugar may be added to the self-assembling peptide solution to improve the osmotic pressure of the solution from hypotonicity to isotonicity, thereby allowing the biological safety to be increased. In certain examples, the sugar may be sucrose or glucose.

The optimal lengths for membrane formation may vary with the amino acid composition. A stabilization factor contemplated by the peptides of the present disclosure is that complementary peptides maintain a constant distance between the peptide backbones. Peptides which can maintain a constant distance upon pairing are referred to herein as structurally compatible. The interpeptide distance can be calculated for each ionized or hydrogen bonding pair by taking the sum of the number of unbranched atoms on the side-chains of each amino acid in the pair. For example, lysine has 5 and glutamic acid has 4 unbranched atoms on its side-chains, respectively.

Peptides, which are not perfectly complementary or structurally compatible, can be thought of as containing mismatches analogous to mismatched base pairs in the hybridization of nucleic acids. Peptides containing mismatches can form membranes if the disruptive force of the mismatched pair is dominated by the overall stability of the interpeptide interaction. Functionally, such peptides can also be considered as complementary or structurally compatible. For example, a mismatched amino acid pair may be tolerated if it is surrounded by several perfectly matched pairs on each side.

The peptides can be chemically synthesized or they can be purified from natural and recombinant sources. Using chemically synthesized peptides may allow the peptide solutions to be deficient in unidentified components such as unidentified components derived from the extracellular matrix of another animal. This property therefore may eliminate concerns of infection, including risk of viral infection compared to conventional tissue-derived biomaterials. This may eliminate concerns of infection including infections such as bovine spongiform encephalopathy (BSE), making the peptide highly safe for medical use.

The initial concentration of the peptide may be a factor in the size and thickness of the membrane, hydrogel, or scaffold formed. In general, the higher the peptide concentration, the higher the extent of membrane or hydrogel formation. Hydrogels, or scaffolds formed at higher initial peptide concentrations (about 10 mg/ml) (about 1.0 w/v percent) may be thicker and thus, likely to be stronger.

Formation of the membranes, hydrogels, or scaffolds may be very fast, on the order of a few minutes. The formation of the membranes or hydrogels may be irreversible. In certain embodiments, the formation may be reversible, and in other embodiments, the formation may be irreversible. The hydrogel may form instantaneously upon administration to a target area. The formation of the hydrogel may occur within about one to two minutes of administration. In other examples, the formation of the hydrogel may occur within about three to four minutes of administration. In certain embodiments the time it takes to form the hydrogel may be based at least in part on one or more of the concentration of the peptide solution, the volume of peptide solution applied, and the conditions at the area of application or injection (for example, the concentration of monovalent metal cations at the area of application, the pH of the area, and the presence of one or more fluids at or near the area). The process may be unaffected by pH of less than or equal to 12, and by temperature. The membranes or hydrogels may form at temperatures in the range of about 1 to 99 degrees Celsius.

The hydrogels may remain in position at the target area for a period of time sufficient to provide a desired effect using the methods and kits of the present disclosure. The desired effect may be to promote bone growth so as to at least partially fill a bone void.

The period of time that the membranes or hydrogels may remain at the desired area may be for one or more days, up to one or more weeks, and up to several months. In other examples, it may remain at the desired area for up to 30 days, or more. It may remain at the desired area indefinitely. In other examples, it may remain at the desired area for a longer period of time, until it is naturally degraded or intentionally removed. If the hydrogel naturally degrades over a period of time, subsequent application or injection of the hydrogel to the same or different location may be performed.

In certain embodiments, the self-assembling peptide may be prepared with one or more components that may provide for enhanced effectiveness of the self-assembling peptide or may provide another action, treatment, therapy, or otherwise interact with one or more components of the subject. For example, additional peptides comprising one or more biologically or physiologically active amino acid sequences or motifs may be included as one of the components along with the self-assembling peptide. Other components may include biologically active compounds such as a drug or other treatment that may provide some benefit to the subject. For example, an antibiotic may be administered with the self-assembling peptide, or may be administered separately.

The peptide, peptide solution, or hydrogel may comprise small molecular drugs to treat the subject or to prevent hemolysis, inflammation, and infection. The small molecular drugs may be selected from the group consisting of glucose, saccharose, purified saccharose, lactose, maltose, trehalose, destran, iodine, lysozyme chloride, dimethylisoprpylazulene, tretinoin tocoferil, povidone iodine, alprostadil alfadex, anise alcohol, isoamyl salicylate, α,α-dimethylphenylethyl alcohol, bacdanol, helional, sulfazin silver, bucladesine sodium, alprostadil alfadex, gentamycin sulfate, tetracycline hydrochloride, sodium fusidate, mupirocin calcium hydrate and isoamyl benzoate. Other small molecular drugs may be contemplated. Protein-based drugs may be included as a component to be administered, and may include erythropoietin, tissue type plasminogen activator, synthetic hemoglobin and insulin.

A component may be included to protect the peptide solution against rapid or immediate formation into a hydrogel. This may include an encapsulated delivery system that may degrade over time to allow a controlled time release of the peptide solution into the target area to form the hydrogel over a desired, predetermined period of time. Biodegradable, biocompatible polymers may be used, such as ethylene vinyl acetate, polyanhydrides, polyglycolic acid, collagen, polyorthoesters, and polylactic acid.

Any of the components described herein may be included in the peptide solution or may be administered separate from the peptide solution. Additionally, any of the methods and methods of facilitating provided herein may be performed by one or more parties.

A peptide, peptide solution, or hydrogel of the disclosure may be provided in a kit. Instructions for administering the solution to a target area of bone in a subject may also be provided in the kit. The peptide solution may comprise a self-assembling peptide comprising between about 7 and about 32 amino acids in an effective amount and in an effective concentration to form a hydrogel to promote bone growth. The instructions for administering the solution may comprise methods for administering the peptide, peptide solution, or hydrogel provided herein, for example, by a route of administration described herein, at a dose, volume or concentration, or administration schedule. The peptide may be amphiphilic and at least a portion of the peptide may alternate between a hydrophobic amino acid and a hydrophilic amino acid.

The kit may also comprise informational material. The informational material may be descriptive, instructional, marketing, or other material that relates to the methods described herein. In one embodiment, the informational material may include information about production of the peptide, peptide solution, or hydrogel disclosed herein, physical properties of the peptide, composition, peptide solution or hydrogel, concentration, volume, size, dimensions, date of expiration, and batch or production site.

The kit may also optionally include a device or materials to allow for administration of the peptide or peptide solution to the desired area. For example, a syringe, pipette, tube, catheter, syringe catheter, or other needle-based device may be included in the kit. Additionally, or alternatively, the kit may include a guidewire, endoscope, or other accompanying equipment to provide selective administration of the peptide solution to the target area.

The kit may comprise in addition to or in the alternative, other components or ingredients, such as components that may aid in positioning of the peptide solution, hydrogel or scaffold. Instructions may be provided in the kit to combine a sufficient quantity or volume of the peptide solution with a sucrose solution that may or may not be provided with the kit. Instructions may be provided for diluting the peptide solution to administer an effective concentration of the solution to the target area. The instructions may describe diluting the peptide solution with a diluant or solvent. The diluant or solvent may be water. Instructions may further be provided for determining at least one of the effective concentration of the solution and the effective amount of the solution to the target area. This may be based on various parameters discussed herein, and may include the dimensions of the target area.

Other components or ingredients may be included in the kit, in the same or different compositions or containers than the peptide, peptide solutions, or hydrogel. The one or more components may include components that may provide for enhanced effectiveness of the self-assembling peptide or may provide another action, treatment, therapy, or otherwise interact with one or more components of the subject. For example, additional peptides comprising one or more biologically or physiologically active sequences or motifs may be included as one of the components along with the self-assembling peptide. Other components may include biologically active compounds such as a drug or other treatment that may provide some benefit to the subject. The peptide, peptide solution, or hydrogel may comprise small molecular drugs to treat the subject or to prevent hemolysis, inflammation, and infection, as disclosed herein. A sugar solution such as a sucrose solution may be provided with the kit. The sucrose solution may be a 20% sucrose solution. Other components which are disclosed herein may also be included in the kit.

In some embodiments, a component of the kit is stored in a sealed vial, for example, with a rubber or silicone closure (for example, a polybutadiene or polyisoprene closure). In some embodiments, a component of the kit is stored under inert conditions (for example, under nitrogen or another inert gas such as argon). In some embodiments, a component of the kit is stored under anhydrous conditions (for example, with a desiccant). In some embodiments, a component of the kit is stored in a light blocking container such as an amber vial.

As part of the kit or separate from a kit, syringes or pipettes may be pre-filled with a peptide, peptide solution, or hydrogel as disclosed herein. Methods to instruct a user to supply a self-assembling peptide solution to a syringe or pipette, with or without the use of other devices, and administering it to the target area through the syringe or pipette, with or without the use of other devices, is provided.

In accordance with one or more embodiments, a kit may include a syringe and a cannula to facilitate administration of the peptide solution. The kit may also include at least one wound dressing to facilitate healing and/or to hold the administered peptide solution in place. One or more materials to be mixed with the peptide solution prior to or during administration may be provided, such as an antibiotic or an anti-inflammatory agent. Other materials may include an allograft or a ceramic material to be mixed with the peptide solution to promote bone growth. An implant may also be included in the kit.

In accordance with one or more embodiments, a kit may include a peptide hydrogel in an effective amount and an effective concentration based at least in part on a dimension of the target site. In some embodiments, the concentration effective to promote bone growth comprises a concentration in a range of about 3 w/v percent to about 5 w/v percent peptide. In at least some embodiments, the peptide hydrogel solution may be substantially non-biologically active. The peptide hydrogel solution may be substantially non-granular. In some embodiments, the self-assembling peptide in the kit comprises about 16 amino acids that alternate between a hydrophobic amino acid and a hydrophilic amino acid. In at least some embodiments, the kit includes Puramatrix® peptide hydrogel. In some embodiments, the pH level of the Puramatrix® peptide hydrogel may be at least about 3, such as about 3.4 or about 3.5.

In accordance with one or more embodiments, the kit may include instructions to use the peptide hydrogel in a bone void filling procedure as discussed herein. The instructions may recite mixing an autograft or an allograft with the peptide solution prior to administration. In some embodiments, the instructions may be directed to a one-step procedure involving administration of the peptide solution. In at least some embodiments, the instructions may direct a practitioner to provide additional doses of the peptide solution subsequent to initial administration and prior to implantation.

In some embodiments of the disclosure, the self-assembling peptides may be used as a coating on a device or an instrument. The self-assembling peptides may also be incorporated or secured to a support, such as gauze or a bandage, or a lining, that may provide a therapeutic effect to a subject, or that may be applied within a target area. The self-assembling peptides may also be soaked into a sponge for use.

In accordance with one or more embodiments, macroscopic structures can be useful for culturing cells and cell monolayers. Cells prefer to adhere to non-uniform, charged surfaces. The charged residues and conformation of the proteinaceous membranes promote cell adhesion and migration. The addition of growth factors, such as fibroblast growth factor, to the peptide macroscopic structure can further improve attachment, cell growth and neurite outgrowth. The porous macrostructure can also be useful for encapsulating cells. The pore size of the membrane can be large enough to allow the diffusion of cell products and nutrients. The cells are, generally, much larger than the pores and are, thus, contained.

In accordance with one or more embodiments, a macroscopic scaffold comprises a plurality of self-assembling peptides, wherein the self-assembling peptides self-assemble into a β-sheet macroscopic scaffold and wherein said macroscopic scaffold encapsulates living cells and wherein said cells are present in said macroscopic scaffold in a three-dimensional arrangement. One or more embodiments also encompass methods of regenerating a tissue comprising administering to a mammal a macroscopic scaffold comprising the disclosed self-assembling peptides at a target site. In at least some embodiments, periodontal tissue is regenerated. In additional embodiments, a scaffold for periodontal tissue regeneration comprises a self-assembling peptide described herein. As used herein in the context of tissue regeneration and/or periodontal tissue regeneration, a scaffold may be a degradable hydrogel.

The function and advantage of these and other embodiments of the methods and kits disclosed herein will be more fully understood from the prophetic example below. The following prophetic example is intended to illustrate the benefits of the disclosed treatment approach, but do not exemplify the full scope thereof.

### Examples

### Prophetic Example

In this prophetic example, a peptide hydrogel may be introduced or administered to a target site. The target site may be an area in which bone growth is desired, or promotion of bone growth is desired. The peptide hydrogel may be introduced or administered to a target site by introducing a delivery device at or near the target site. An end of the delivery device may be positioned at or near, for example, proximate, the target site. A solution comprising a self-assembling peptide may be administered through the delivery device to the target site. It is expected that the solution will form a hydrogel, for example, a hydrogel scaffold, under physiological conditions to promote bone growth. The peptide solution may comprise a self-assembling peptide comprising between about 7 and about 32 amino acids. The peptide solution may be delivered in an effective amount and in an effective concentration to form a hydrogel, for example, hydrogel scaffold under physiological conditions to promote bone growth. After administration, the delivery device may be removed from being at or near, for example, proximate, the target site.

The peptide hydrogel may be PuraMatrix® or PuraMatrix Plus™ peptide solutions, which are peptide solutions in water comprising a synthetic, 16-amino acid polypeptide with a repeating sequence of arginine, alanine, and aspartic acid, or RADARADARADARADA (RADA16). PuraMatrix® peptide solution may be in a peptide solution having a pH between about 2 to about 3. PuraMatrix Plus™ peptide solution may be in a peptide solution having a pH level of about 3.4 or greater, such as about 3.5. In other embodiments, the peptide hydrogel may be another self-assembling peptide such as KLDL12 (or KLD12) having the sequence Lys-Leu-Asp-Leu-Lys-Leu-Asp-Leu-Lys-Leu-Asp-Leu (KLDL)₃. In accordance with one or more further embodiments, the peptide hydrogel may be IEIK13 having the sequence Ile-Glu-Ile-Lys-Ile-Glu-Ile-Lys-Ile-Glu-Ile-Lys-Ile (IEIK)₃I. Other peptide hydrogels exhibiting similar beneficial properties as discussed herein may also be used. Any of these peptide hydrogels may have a pH level of greater than about 3.0, such as about 3.4 or 3.5.

The peptide hydrogels, such as RADA16, KLD12, or IEIK13, including PuraMatrix® or PuraMatrix Plus™, may be present at a concentration effective to promote bone growth under physiological conditions. This concentration may be in a range of about 0.1 w/v percent to about 5 w/v percent peptide. Specifically, the concentration may be in a range of about 1 w/v percent to about 5 w/v percent peptide. More specifically, the concentration may be in a range of about 3 w/v percent to about 5 w/v percent peptide.

A procedure to promote bone growth may be performed using one or more of the peptide hydrogels discussed above. The procedure may be performed on non-weight bearing, or weight bearing bones, for example, in non-weight bearing or weight bearing healing models.

A peptide hydrogel solution comprising about 1 percent to about 5 percent peptide may be introduced to the target area. The amount used may be determined based on the size of the bone defect. For example, if the bone defect (for example, bone void) has a volume of approximately 1 cm³, then the amount of the peptide solution used may be approximately equal to that volume. In some embodiments approximately 1.2 times, 1.5 times, 1.7 times, or 2.0 times the volume of the bone void may be introduced to the target site.

The testing may comprise comparing introduction of the peptide hydrogel solution of the present disclosure to other implantable materials, for example, Collaplug Collagen Implant, Collagraft Strip Implant, Tricalcium Phosphate, or demineralized freeze dried bone allograft (DFDBA). These conventional implants may be used in accordance with the manufacturer's instructions.

The testing may also comprise comparison of the peptide hydrogel solutions to one another. For example, the testing may comprise comparing PuraMatrix® to at least one of PuraMatrix Plus™, KLD12, and IEIK13. In specific testing PuraMatrix® may be compared to PuraMatrix Plus™.

The testing may also comprise comparison of one or more peptide hydrogels at the same concentrations of peptide. For example, a 1% peptide solution of PuraMatrix® may be compared to at least one of a 1% peptide solution of PuraMatrix Plus™ KLD12, and IEIK13. In another aspect of the testing, a 3% peptide solution of PuraMatrix® may be compared to at least one of a 3% peptide solution of PuraMatrix Plus™, KLD12, and IEIK13. In another aspect of the testing, a 5% peptide solution of PuraMatrix® may be compared to at least one of a 5% peptide solution of PuraMatrix Plus™, KLD12, and IEIK13.

The testing may also comprise comparison of one or more peptide hydrogels at different concentrations of peptide. For example, one or more of a 1% peptide solution of PuraMatrix®, PuraMatrix Plus™, KLD12, and IEIK13 may be compared to one or more of a 3% peptide solution or a 5% peptide solution of PuraMatrix®, PuraMatrix Plus™, KLD12, and IEIK13.

One or more control tests may be done in which no implant is used for comparison with the peptide hydrogel and the other implants.

Blood or saline may be applied with any one or more of the peptide hydrogel solutions.

Additional peptide hydrogel solution may be introduced to the target site at any time after the initial administration of the solution. This peptide hydrogel solution may be the same peptide or different peptide, or same concentration or different concentration as the peptide hydrogel solution initially administered.

It may be found that the peptide hydrogel solutions, upon application, may form peptide hydrogel scaffolds. The peptide hydrogel scaffolds may promote bone growth to provide bone growth or ingrowth, and promote healing of the target area. The peptide hydrogel scaffolds may promote bone growth to provide bone growth or ingrowth, and promote healing of the target area in a superior manner when compared to the control and other implantable materials, which may provide one or more of less bone growth, less healing, fibrous scar tissue, vascular scar tissue, and discontinuous healing.

In certain aspects, it may be found that the peptide hydrogel scaffolds at an elevated pH and/or elevated concentration, including of PuraMatrix Plus™, may promote bone growth to a greater extent than that of one or more other peptide hydrogels, namely, one or more of PuraMatrix®, KLD12, and IEIK13. It may also be found that PuraMatrix Plus™ may have greater mechanical strength, higher levels of biocompatibility, and more vital bone growth in comparison to one or more of the other peptide hydrogels tested.

The bone growth or ingrowth may occur over a period of time, for example, a predetermined period of time. The period of time or predetermined period of time may be based on the size of the target area (for example, length, width, depth, volume), the location of the target area, and the health of the subject.

Evaluation of Results

Biopsies (for example, bone core samples) may be taken during the testing or at the end of the testing to evaluate samples through conventional hematoxylin-eosin (H&E) techniques. Samples may be evaluated for histologic and histomorphometric analysis.

The analysis may be performed using an optical microscope with an inverted digital camera. At least two slides of each height level per bone core specimen may be analyzed. Images of the samples may be captured at the same magnification. Quantification of the percent vital bone, remaining graft particle, and non-mineralized connective tissue may be performed using specialized software. Vital bone may generally be associated with or defined by the identification of osteocytes in the lacunae.

As an additional measure of efficacy, Cone Beam Computational Tomography (CBCT) scans may be evaluated at the end of the study in a blinded fashion. Transverse sections of the sites may be evaluated to measure the change in height and width of the bone between baseline and post augmentation.

It is expected that all cores will show minimal inflammatory cell infiltration consistent with resorbing graft particles or material and normal bone turnover. It is expected that no abscess formation will be observed in any of the cores evaluated. It may be expected that the peptide hydrogel solution may show greater new bone formation at the target sites than other implantable materials. Particularly, it may be expected that the PuraMatrix Plus™ may show greater new bone formation at the target sites than other implantable materials, including other peptide hydrogels.

The size of the bone marrow spaces is expected to be consistent with new bone in the peptide hydrogel cores.

It may be observed that the peptide hydrogels offer advantages over other implant materials in terms of handling and surgical technique. Considerably less time may be necessary to prepare the graft. It may be found that the peptide hydrogels may be easy to apply, perfectly filling the surgical site, requiring less exposure time for the surgical site and therefore minimizing risk of contamination. It may be expected that due to higher mechanical strength and other properties of PuraMatrix Plus™ that PuraMatrix Plus™ may be easier to handle, easier to fill, and be maintained within the surgical site or bone void to provide superior results to other conventional techniques, and other peptide hydrogels.

It is expected that all subjects tested with the peptide hydrogels will show serum IgG results within the normal range.

It is expected that the percentage of vital bone may be greater in the target areas in which a peptide hydrogel solution is introduced than in those in which other implantable materials, or no material is introduced. It may be expected that PuraMatrix Plus™ may have a greater percentage of vital bone in the target areas as compared to other peptide hydrogel solutions such as PuraMatrix® or other implantable materials.

Radiographic evaluations may be performed to three-dimensionally evaluate bone height and width changes following a procedure as described herein.

It is expected that images show significant changes in bone height for most of the subjects treated with the peptide hydrogel solutions. It may be expected that PuraMatrix Plus™ may have a greater change in bone height in the target areas as compared to other peptide hydrogel solutions such as PuraMatrix® or other implantable materials.

### CONCLUSIONS

This example may show that peptide hydrogel solutions can be safely and successfully used in bone growth procedures. An efficacy objective may be met by showing that the formation of new vital bone is better than, or similar to that observed for the control treatment. A supplemental efficacy objective may be met by showing that, for peptide hydrogel solutions, and the control treatments, implants placed in the graft were successful after a predetermined period of time.

Additionally, it may be found that more time is necessary to prepare the graft (about 15 minutes, with dehydration and waiting time) when using DFDBA, or another implant material, versus the peptide hydrogel solution. It may be difficult to predict the exact amount needed, and therefore, it may take more time to prepare an additional graft, if needed. More time may also be needed with DFDBA and other implant materials to condense the graft in the augmented area. More care is also needed to carefully transfer the graft into the site. There may be more contamination risk or risk for loss of graft during the transfer.

With peptide hydrogel solutions, it may be found that considerably less time is necessary to prepare the graft (about 2-5 minutes, no dehydration or waiting time). It may be easy to predict the exact amount needed, and if an additional amount is needed, it takes only 1-2 minutes to add a new syringe containing the peptide hydrogel solution. There is almost no extra time needed to condense the graft in the target area. Additionally, less contamination risk and less care is needed to transfer the graft into the surgical site.

It is also expected to be found that peptide hydrogels are easy and quick to apply. Therefore there is less exposure time for the surgical site. It may perfectly fill the surgical site, and there is no contamination risk or risk of loss of material. There is also no post-operative problems or clinical evidence of any intra-oral or extra-oral pathology.

It may also be expected that PuraMatrix Plus™ may have superior properties to other peptide hydrogels such as PuraMatrix®. This may include greater mechanical strength, higher levels of biocompatibility, and more vital bone growth in comparison to one or more of the other peptide hydrogels tested.

It may also be expected that higher concentrations, for example, in a range of about 3% to 5% peptide solutions may also exhibit superior qualities as compared to other peptide solutions at lower percentages of peptides.

Various embodiments of the materials and methods discussed herein are not limited in their application to the details as set forth in the description. One or more embodiments are capable of being practiced or carried out in various ways beyond those exemplarily presented herein.

What is claimed is:

1. A method of filling a bone void in a subject, comprising:
introducing a delivery device to a bone of a subject;
positioning an end of the delivery device proximate a void in the bone where promotion of bone growth is desired;
administering through the delivery device a solution comprising a self-assembling peptide comprising between about 7 and about 32 amino acids in an effective amount and in a concentration in a range of about 3 w/v percent to about 5 w/v percent peptide to form a hydrogel scaffold under physiological conditions to promote bone growth at the target site; and
removing the delivery device from the subject.

2. The method of claim 1, wherein the self-assembling peptide comprises about 16 amino acids that alternate between a hydrophobic amino acid and a hydrophilic amino acid.

3. The method of claim 2, wherein the peptide in the solution comprises RADA16.

4. The method of claim 1, wherein the peptide in the solution comprises IEIK13 or KLD12.

5. The method of claim 1, wherein the peptide solution is substantially non-biologically active.

6. The method of claim 1, further comprising mixing the peptide solution with an autograft or an allograft prior to administration.

7. The method of claim 1, wherein the method is used after a surgical procedure.

8. The method of claim 1, further comprising applying a wound dressing at the target site after administration of the peptide solution.

9. The method of claim 1, further comprising administering a supplemental volume of the peptide solution at the target site after a predetermined period of time.

10. The method of claim 1, further comprising visualizing the target site after a predetermined period of time to assess bone augmentation.

11. The method of claim 1, wherein the hydrogel scaffold comprises nanofibers having a diameter of about 10 nanometers to about 20 nanometers.

12. The method of claim 1, wherein the bone is alveolar bone.

13. The method of claim 1, wherein the peptide solution has a pH level of at least about 3.4.

14. A method of filling a bone void in a subject, comprising:
introducing a delivery device to a bone of a subject;
positioning an end of the delivery device proximate a void in the bone where promotion of bone growth is desired;
administering through the delivery device a solution having a pH level of between about 3.0 and about 3.5, the solution comprising a self-assembling peptide comprising between about 7 and about 32 amino acids in an effective amount and in a concentration sufficient to form a hydrogel scaffold under physiological conditions to promote bone growth at the target site; and
removing the delivery device from the subject.

15. The method of claim 14, wherein the self-assembling peptide comprises about 16 amino acids that alternate between a hydrophobic amino acid and a hydrophilic amino acid.

16. The method of claim 15, wherein the peptide in the solution comprises RADA16.

17. The method of claim 14, wherein the peptide in the solution comprises IEIK13 or KLD12.

18. The method of claim 14, wherein the peptide solution is substantially non-biologically active.

19. The method of claim 14, further comprising mixing the peptide solution with an autograft or an allograft prior to administration.

20. The method of claim 14, wherein the method is used after a surgical procedure.

21. The method of claim 14, further comprising applying a wound dressing at the target site after administration of the peptide solution.

22. The method of claim 14, further comprising administering a supplemental volume of the peptide solution at the target site after a predetermined period of time.

23. The method of claim 14, further comprising visualizing the target site after a predetermined period of time to assess bone augmentation.

24. The method of claim 14, wherein the hydrogel scaffold comprises nanofibers having a diameter of about 10 nanometers to about 20 nanometers.

25. The method of claim 14, wherein the bone is alveolar bone.

26. A kit for filling a bone void in a subject, comprising:
a solution comprising a self-assembling peptide comprising between about 7 amino acids and about 32 amino acids in an effective amount and in an effective concentration to form a hydrogel scaffold under physiological conditions to promote bone growth at a target site; and
instructions for administering the solution to the target site in a bone of the subject.

27. The kit of claim 26, wherein at least one of the effective amount and the effective concentration is based in part on a dimension of the target site.

28. The kit of claim 26, wherein the concentration effective to promote alveolar bone growth comprises a concentration in a range of about 3 w/v percent to about 5 w/v percent peptide.

29. The kit of claim 26, wherein the peptide solution has a pH level of between about 3.0 and about 3.5.

30. The kit of claim 26, wherein the peptide solution has an osmolality level of between about 0 and about 300 mOsm/L.

31. The kit of claim 26, wherein the peptide in the solution comprises RADA16, KLD12, or IEIK13.

32. The kit of claim 26, wherein the peptide solution is substantially non-biologically active.

33. The kit of claim 26, wherein the peptide solution comprises at least one of an antibiotic and an anti-inflammatory agent.

34. The kit of claim 26, further comprising a ceramic or an allograft to be mixed with the peptide solution prior to administration.

35. The kit of claim 26, further comprising at least one of a syringe and a cannula to facilitate administration of the peptide solution.

36. The kit of claim 26, further comprising a wound dressing.
